(19) United States
(12) Patent Application Publication
(10) Pub. No.: US 2002/0137938 A1
(43) Pub. Date: Sep. 26, 2002

(54) Novel process

(75) Inventor: Edward Lucas, Ayrshire (GB)

Correspondence Address: GLAXOSMITHKLINE, Corporate Intellectual Property - UW2220, P.O. Box 1539, King of Prussia, PA, 19406-0939, US

(73) Assignee: SmithKlineBeecham plc

(21) Appl. No.: 10/109,119

(22) Filed: Mar. 27, 2002

Related U.S. Application Data
(63) Continuation of application No. 09/635,545, filed on Aug. 10, 2000, now abandoned.
Application No. 09/635,545 is a continuation of application No. 09/370,041, filed on Aug. 6, 1999, now abandoned.

(30) Foreign Application Priority Data
Aug. 7, 1998 (GB) 9817195.2

(51) Int. Cl. C 07D 4 7/02

(52) U.S. Cl. 546/197

(57) ABSTRACT

A process for the preparation of a carbamate of formula (1):

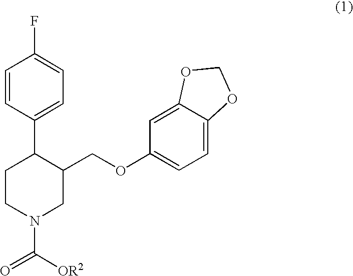

in which R² is a C₁₋₆ alkyl group, optionally substituted by halogen, a C₃₋₆ cycloalkyl group, aralkyl or optionally substituted aryl group; comprises reacting a solution of a compound of formula (2)

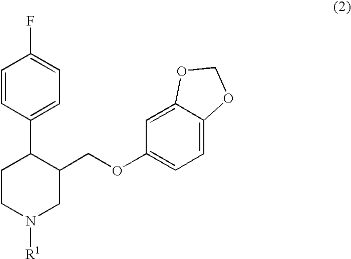

in which R¹ is an alkyl, arylalkyl or alkynyl group; at a temperature in the range of 50-100° C. with a haloformate of formula Hal-CO₂R².

The present invention relates to a new process for preparing pharmaceutically active compounds and intermediates therefor.

Pharmaceutical products with antidepressant and anti-Parkinson properties are described in U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylenedioxy-phenoxymethyl)-piperidine. This compound is used in therapy as the hydrochloride salt to treat inter alia depression, obsessive compulsive disorder (OCD) and panic.

Previously published processes to paroxetine utilise as a key intermediate the compound

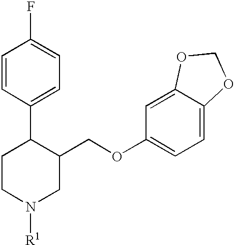

in which the piperidine nitrogen is protected by a group R¹, usually an alkyl (typically methyl) group. The N-protected piperidine must be converted to the free base or a salt thereof.

In U.S. Pat. No. 4,007,196, Example 2, a solution of 4-fluorophenyl-3-(3′,4′-methylenedioxyphenoxymethyl)-1-methyl piperidine in dichloromethane was treated with phenyl chloroformate in dichloromethane at 0-5° C. After leaving overnight, the solution was washed with 1M NaOH and then 1M HCl, dried and evaporated. The solid residue was suspended in benzene, filtered and evaporated. The evaporation residue was heated at reflux with KOH and 2-methoxyethanol for 4 hours and then evaporated. Water was added and the mixture extracted with benzene, dried, and evaporated to give the N-deprotected compound.

In Example 8 of EP 0 152 273, 4-(4′-fluorophenyl)-3-(4′-methoxyphenoxymethyl)-1-methyl piperidine is dissolved in toluene and treated at 0° C. with a solution of 1.9 equivalents of phenyl chloroformate in toluene over 30 minutes. The mixture was allowed to stand at room temperature for 20 hours. A further 1.9 equivalents of phenyl chloroformate were added and the mixture left for 72 hours. The solution was washed 2N NaOH, then water, then 1N HCl and finally saturated aqueous NaCl. The organic phase was dried and concentrated to give an oil which was then crystallised as a white crystals from 96% ethanol. This intermediate was mixed with KOH and 2-methoxyethanol and stirred at 130-140° C. for 4 hours and partitioned between water and toluene. The organic phase was dried and evaporated to give the N-deprotected compound as an oil which was then converted to the acetate salt.

EP-0 810 225 develops these processes a little further; proposing that 4-(4′-fluorophenyl)-3-(3′,4′-methylenedioxyphenoxymethyl)-1-methyl piperidine is converted to the 1-methoxycarbonyl or 1-ethoxycarbonyl derivative using the methyl or ethyl ester of chloroformic acid and then hydrolysed to paroxetine itself. This publication suggests using a wide range of solvents at a range of temperatures from 10-150° C. for the preparation of the carbamate. However, only toluene and tetrahydrofuran are exemplified as solvents. In every example, the chloroformate is added whilst cooling in ice and only when the solvent is THF is it suggested that the reaction mixture can then be heated. Even so, reaction times range from 7 hours to 3 days. Where the solvent is toluene, the inventors of EP-0 810 225 preferred to follow the teaching of EP-0 152 273 above, adding the chloroformate at 0° C. and then stirring the reaction mixture at room temperature overnight. It is indicated that the reaction only proceeds satisfactorily in the presence of a base. This publication specifically teaches against the use of phenyl chloroformate at this stage in the synthesis of paroxetine on the grounds of a low conversion to the phenyl carbamate and difficulty in separating the starting materials from the carbamate leading to low yields. The carbamate is hydrolysed by treatment with KOH in toluene at reflux or ethanol at 90° C. for periods of 2-3 days.

These processes are not applicable to implementation on an industrial scale. The applicant's investigations have led to surprising modifications to the reaction conditions suitable for industrial scale production.

In its broadest sense, the present invention provides a process for the preparation of a carbamate of-formula (1):

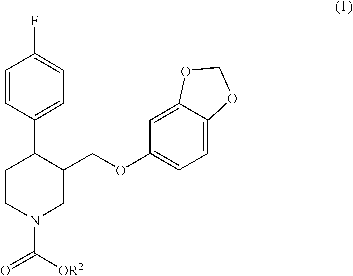

in which R² is a C₁₋₆ alkyl group optionally substituted by halogen, a C₃₋₆ cycloalkyl group, arylalkyl, or optionally substituted aryl. Suitable examples include phenyl, trichloroethyl, and 1-chloroethyl.

The method comprises treating a solution of a compound of formula (2)

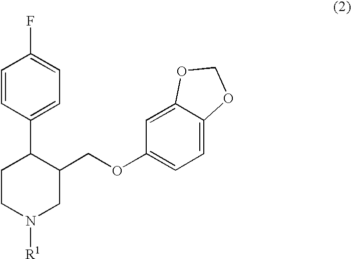

in which R¹ is a C₁₋₆ alkyl, aralkyl or alkynyl group, preferably methyl, at a temperature of 50-100° C., with a haloformate of formula Hal-CO₂R².

Suitably the reaction temperature is about 55-75° C., preferably about 60-70° C., more preferably at 63° C.±5° C.

Preferably, the haloformate is phenyl chloroformate.

Preferably, the solvent is toluene, benzene, xylene, dichloromethane, or 1,2-dichloroethane. Most preferably the solvent is toluene.

The compound of formula (2) may include solvent residues, particularly methanol and/or isopropanol, but preferably these are present in an amount of not more than 0.05% w/w. In addition, the water content of the toluene solution should preferably be less than 0.05% w/w. Accordingly, the toluene is preferably dried, for example by azeotropic distillation. Alternatively, or additionally, these levels may be conveniently achieved by heating the compound of formula (2) in the reaction solvent to reflux at around 65° C. under reduced pressure or at atmospheric pressure to remove the solvents by distillation. Dean & Stark conditions have also been successfully used.

Preferably the chloroformate is free of HCl and water. Whilst following the prior art procedures the chloroformate can be added as a solution in toluene, preferably, it is added undiluted.

Whilst EP-0 810 225 indicates that the presence of a base is essential for a successful conversion to the carbonate, this has not been found to be the case under the reaction conditions outlined above, certainly with organic bases which have led to lower yields and purities. Whilst not seeking to exclude the use of bases, their presence has been found to be largely unnecessary.

Under the conditions described above, the reaction is very fast, typically being complete within about 1-2 hours. This is a considerable advantage over the prior art which mentions periods of 1-3 days in toluene. Yields are also very much improved, being in the order of 80-95%, compared with the 33% yield of EP-0 810 225.

In a modification of this process, phenyl chloroformate is added to a solution in toluene of a compound of formula (2); and the mixture is heated at the same temperatures as described above.

Whilst EP-0 810 225 merely filters and evaporates the reaction mixture to produce the crude carbamate, U.S. Pat. No. 4,007,196 and EP 152 273 wash the solution with aqueous NaOH and then dilute HCl before drying and evaporating to obtain the carbamate. It has been found that after cooling to around 20° C. (room temperature), quenching with 10% aq. sulphuric acid provides surprising advantages. It removes unreacted starting material (compound of formula (2)) more efficiently than hydrochloric acid. It is also effective at hydrolysing unreacted phenyl chloroformate so affording a cheap and effective one step purification, ideally suited to commercial manufacture. This process step is more widely applicable to paroxetine analogues generally, for example for treating crude carbamates obtained by routes other than the process of this invention, as in the prior art documents acknowledged above, and forms another aspect of the present invention.

In addition, the sulphate of the starting compound (formula (2)) is insoluble in both the organic and inorganic phases. As such, one would not anticipate that excess starting material could be removed by the quenching step described above. However, it has been found that the sulphate is, in fact, associated with the aqueous phase, as a distinct solid phase and so can be easily removed, for example, by phase separation.

The toluene phase is then washed (twice) with water at ambient temperature—a suitable volume for each water wash is between 1:10 of the toluene volume and 1:2, preferably between 1:5 and 1:8, optionally dried, and filtered, preferably with use of a filter agent such as celite.

Trans-(−)-4-(4-fluorophenyl)-3-(3′,4′-methylenedioxyphenoxymethyl)-1-phenoxy carbonylpiperidine is a difficult compound to isolate in a pure form and no satisfactory isolation method has been provided in existing publications. Distillation under vacuum removes the toluene and leaves an oil or unmanageable solid lump, and such processes can only be used on the laboratory scale. However efficient removal of solvent from an oil is difficult to achieve on the manufacturing scale, and is time consuming and expensive. The resulting oil or lump is difficult to remove from the distillation vessel and is not suitable for storage as an intermediate. In an industrial context, this means that stockpiling is not conveniently possible, and this greatly reduces the flexibility of plant utilisation.

It has now been determined that a crystalline form of the product can be prepared and this overcomes the disadvantages mentioned above. After most of the toluene has been removed (complete removal of toluene is no longer necessary), hot propan-2-ol is added to dissolve the residue, and the solution cooled to give a crystalline solid with improved purity and handling properties. A similar result can be achieved by progressively replacing toluene by fresh propan-2-ol during the course of the azeotropic distillation. This variation offers an advantage in that it allows manufacturing apparatus to be used which can only remove a smaller proportion of the total solvent. Crystallisation from propan-2-ol provides a mobile slurry suitable for transfer and filtration using convenient industrial apparatus. Ethanol or denatured industrial methylated spirits may also be used.

The process of this invention may be used to prepare active compounds described in U.S. Pat. No. 3,912,743 and U.S. Pat. No. 4,007,196, and preferably to prepare paroxetine by conventional removal of the carbamate for N-deprotection, as described above.

Paroxetine is preferably obtained as or converted to a pharmaceutically acceptable salt, such as the methanesulfonate, or the hydrochloride salt and most preferably the hemihydrate of that salt. The present invention includes within its scope the compound paroxetine and its salts, particularly paroxetine hydrochloride, especially as the hemihydrate or anhydrate, when obtained via any aspect of this invention, and any novel intermediates resulting from the described procedures. The preparation of paroxetine hydrochloride as a crystalline hemihydrate is disclosed in EP-A-0223403 (Beecham Group), and various crystalline anhydrate forms are disclosed in WO 96/24595 (SmithKline Beecham).

Paroxetine is the (−)-trans isomer of 4-(4′-fluorophenyl)-3-(3′,4′-methylenedioxy-phenoxymethyl)-piperidine. Following the procedure of EP-0 152 273, optical resolution may be carried out prior to coupling with the phenol. Alternatively, resolution may be carried out at other stages, such as after deprotection of the piperidine nitrogen. Example 4 describes two suitable methods of resolution of the N-deprotected compound.

Paroxetine obtained using this invention may be formulated for therapy in the dosage forms described in EP-A-0223403 or WO96/24595, either as solid formulations or as solutions for oral or parenteral use.

Therapeutic uses of paroxetine, especially paroxetine hydrochloride, obtained using this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the Disorders”.

Accordingly, the present invention also provides:

a pharmaceutical composition for treatment or prophylaxis of the Disorders comprising paroxetine or a salt such as paroxetine hydrochloride obtained using the process of this invention and a pharmaceutically acceptable carrier,

the use of paroxetine or a salt such as paroxetine hydrochloride obtained using the process of this invention to manufacture a medicament for the treatment or prophylaxis of the Disorders; and

a method of treating the Disorders which comprises administering an effective or prophylactic amount of paroxetine or a salt such as paroxetine hydrochloride obtained using the process of this invention to a person suffering from one or more of the disorders.

Most suitably a compound obtainable by the process of the invention is applied to the treatment of depression, OCD and panic, typically formulated into a pharmaceutical composition with conventional excipients.

The compositions of this invention are usually adapted for oral administration, but formulations for dissolution for parental administration are also within the scope of this invention.

The composition is usually presented as a unit dose composition containing from 1 to 200 mg of active ingredient calculated on a free base basis, more usually from 5 to 100 mg, for example 10 to 50 mg such as 10, 12.5, 15, 20, 25, 30 or 40 mg by a human patient. Most preferably unit doses contain 20 mg of active ingredient calculated on a free base basis. Such a composition is normally taken from 1 to 6 times daily, for example 2, 3 or 4 times daily so that the total amount of active agent administered is within the range 5 to 400 mg of active ingredient calculated on a free base basis. Most preferably the unit dose is taken once a day.

Preferred unit dosage forms include tablets or capsules.

The compositions of this invention may be formulated by conventional methods of admixture such as blending, filling and compressing.

Suitable carriers for use in this invention include a diluent, a binder, a disintegrant, a colouring agent, a flavouring agent and/or preservative. These agents may be utilized in conventional manner, for example in a manner similar to that already used for marketed anti-depressant agents.

Specific examples of pharmaceutical compositions include those described EP-B-0-223403, and U.S. Pat. No. 4,007,196 in which the products of the present invention may be used as the active ingredients.

This invention is illustrated by the following Examples.

### EXAMPLE 1

4-(4-fluorophenyl)-1-phenoxycarbonyl-3-(3′,4′-methylenedioxyphenoxymethyl)-piperidine

4-(4-fluorophenyl)-1-methyl-3-(3′,4′-methylenedioxyphenoxymethyl)piperidine (100 g, 96% purity, 0.279 mol) was dissolved in dry toluene (750 ml) and azeotropically concentrated to 500 ml. A further 250 ml toluene was added and again azeotropically concentrated to 500 ml. The solution was then heated to 60-65° C. with stirring, and phenyl chloroformate (39.5 ml, 0.307 mol, 1.1 equiv) was added over 15 minutes and the mixture stirred for 1 hour at 60-65° C. The mixture was then cooled to 20° C. and washed with 10% sulphuric acid (125 ml). The acid washes were extracted with toluene (50 ml) and the organic phases combined, washed (water, 100 ml), dried (MgSO₄, 20 g), filtered using Celite and washed twice with toluene (2×50 ml). The solution was concentrated and propan-2-ol (125 ml) added. The solution was re-concentrated, further propan-2-ol (125 ml) added, again re-concentrated and further propan-2-ol (400 ml) added. After ensuring that ail the material had fully dissolved, the solution was cooled to 0-5° C. slowly over the course of 2 hours and then stirred at this temperature for about 1 hour, whereupon the product crystallised. This was removed by filtration (with washing—cold propan-2-ol, 2×75 ml), and dried to give the title compound (107 g, 0.245mol, 88%).

Infra Red Spectrum (Nujol Mull)

Bands at: 1711, 1625, 1504, 1486, 1396, 1273, 1222, 1187, 1161, 1134, 1106,1040, 1020, 832, 788, 751, 695, 548 cm⁻¹

### EXAMPLE 2

Phenyl chloroformate (72 ml) is slowly added to a solution of 4-(4-fluorophenyl)-1-methyl-3-(3′,4′-methylenedioxyphenoxymethyl)piperidine (180 g) in toluene (1400 ml) keeping the temperature between 60-65° C. and the mixture is stirred for a further 1-2 hours. The mixture is washed with 10% aqueous sulphuric acid (250 ml) and then with water (2×300 ml). The aqueous liquors are separated and washed with toluene (200 ml). The combined toluene extracts are filtered through a filter aid, such as celite. The solution is concentrated and the solvent replaced by propan-2-ol (final volume 1 liter). The mixture is cooled to below 5° C., the product is isolated, washed with propan-2-ol and dried.

Typical Yield: 80%

### EXAMPLE 3

Industrial Scale Manufacture of 4-(4-fluorophenyl)-1-phenoxycarbonyl-3-(3′,4′-methylenedioxyphenoxymethyl)piperidine

Toluene (1000 L) and 4-(4-fluorophenyl)-1-methyl-3-(3′,4′-methylenedioxy-phenoxymethyl)piperidine (180 kg) are charged to a reaction vessel, stirred and brought to reflux under reduced pressure to ensure dryness. The temperature of the mixture is stabilized at 60-65° C. and phenyl chloroformate (72 kg) is added while maintaining stirring at the specified temperature. After 1 hour, the vessel contents are cooled to 20° C. and quenched with 10% aqueous sulphuric acid (250 L). The layers are allowed to separate, and the aqueous layer is discarded to waste. Water (200 L) is added with stirring to the toluene layer, and once again the layers are allowed to separate, and the aqueous layer is discarded to waste. Celite (5 kg) is added to the stirred toluene layer, and the mixture filtered into a vacuum distillation vessel, where the toluene is removed to leave an oil at a final temperature of 65-70° C. Propan-2-ol (1000 L) at 65° C. is charged to the vessel, stirred to dissolve the oil, and removed by reduced pressure distillation until a white solid remains. A further quantity of propan-2-ol (1000 L) at 65° C. is charged to the vessel, and the slurry transferred to a crystallizer where it is cooled to approximately 0° C. and stirred at that temperature for 2 hours. The product is then filtered, dried under vacuum, and kegged.

### EXAMPLE 4

trans-(−)-1-ethyloxycarbonyl-4-(4-fluorophenyl)-3-((3′,4′-methylenedioxyphenyl)oxymethyl)piperidine

A solution of trans-(−)-1-benzyl-4-(4-fluorophenyl)-3-((3′,4′-methylenedioxyphenyl)oxymethyl)piperidine (0.857 g) was dissolved in 1,2-dichloroethane (10 ml) and the solution treated with ethyl chloroformate (0.23 ml) under argon. The mixture was stirred and heated at reflux for 4 hours, cooled, then added to a stirred mixture of 0.5 molar aqueous hydrochloric acid (20 ml) and dichloromethane (20 ml). The organic phase was separated, dried over anhydrous magnesium sulphate, and evaporated to the required product, as an oil (0.817 g).

### EXAMPLE 5

trans-(−)-1-(1-chloroethyloxycarbonyl)-4-(4-fluorophenyl)-3-((3′,4′-methylenedioxyphenyl)oxymethyl)piperidine

A solution of trans-(−)-1-benzyl-4-(4-fluorophenyl)-3-((3′,4′-methylenedioxyphenyl)oxymethyl)piperidine (1.07 g) was dissolved in 1,2-dichloroethane (10 ml) and the solution cooled below 5° C., then treated with 1-chloroethyl chloroformate (0.3 ml). The mixture was stirred and heated at reflux for 2 hours, cooled, diluted with dichloromethane (20 ml), then stirred with 0.5 molar aqueous hydrochloric acid (20 ml). The organic phase was separated, dried over anhydrous magnesium sulphate, and evaporated to the required product, as a crystallizing oil.

### EXAMPLE 6

trans-(−)-1-phenyloxycarbonyl-4-(4-fluorophenyl)-3-((3′,4′-methylenedioxyphenyl)oxymethyl)piperidine

A solution of trans-(−)-1-benzyl-4-(4-fluorophenyl)-3-((3′,4′-methylenedioxyphenyl)oxymethyl)piperidine (0.97 g) was dissolved in 1,2-dichloroethane (12 ml) and the solution treated with phenyl chloroformate (0.32 ml) under nitrogen. The mixture was stirred and heated at reflux for 3 hours, cooled, then added to a stirred mixture of 0.5 molar aqueous hydrochloric acid (15 ml) and dichloromethane (20 ml). The organic phase was separated, dried over anhydrous magnesium sulphate, and evaporated to the required product, as an oil. Part of the oil was crystallized from propan-2-ol (0.53 g).

### EXAMPLE 7

Preparation of Paroxetine Hydrochloride by the Resolution of (±) trans 4-(4-fluorophenyl)-3-(3′4′-methylenedioxyphenoxymethyl)piperidine.

i) (±) trans 4-(4′-fluorophenyl)-3-(3′4′-methylenedioxyphenoxymethyl) piperidine (1.0 g) was dissolved in methanol (10 ml) and added to a solution of L(−)-di-p-toluoyl tartaric acid (1.25 g) in methanol (10 ml). The mixture was seeded and allowed to stand at room temperature and the crystalline product examined by chiral HPLC, using the following system:

| Column: | Chiralpak AD (Diacel Chemical Industries) |
|---|---|
| Dimensions/particle size: | 250 × 4.6 mm, 10 um |
| Eluent: | Hexane/Ethanol/Trifluoroacetic acid |
| | 88:12:0.06 |
| Eluent flow rate: | 1 ml/minute |
| Detection: | UV at 295 nm |
| Column temperature: | 25° C. |
| Injection volume: | 20 microliter |
| Conditions: | Isocratic |
| Sample preparation: | 0.3 mg/ml in hexane/ethanol/methanol |
| | 80:10:10 |

Chiral HPLC analysis confirmed that substantially pure (−) trans L(−)-di-p-toluoyl tartrate salt had been isolated. The salt may be further purified by recrystallisation from methanol.

ii) (±) trans 4-(4′-fluorophenyl)-3-(3′4′-methylenedioxyphenoxymethyl) piperidine (0.50 g) was dissolved in acetonitrile (10 ml) and added to a solution of L (−)-dibenzoyl tartaric acid (0.65 g) in acetonitrile (10 ml). The mixture was seeded and stirred at room temperature. The crystalline product was shown by chiral HPLC to be significantly enriched with the (−) trans dibenzoyl tartrate salt.

iii) Paroxetine free base is liberated from the (−) trans 4-(4′-fluoro-phenyl)-3-(3′4′-methylenedioxyphenoxymethyl) piperidine di-p-toluoyl or dibenzoyl tartrate salt by stirring in a mixture of toluene and dilute aqueous sodium hydroxide. The phases are separated and the toluene phase washed with water. Concentrated aqueous hydrochloric acid is then added and the crystalline precipitate collected by filtration and dried.

1. A process for the preparation of a carbamate of formula (1): 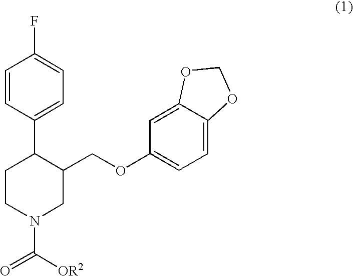
in which R² is a C₁₋₆ alkyl group, optionally substituted by halogen, a C₃₋₆ cycloalkyl group, aralkyl or optionally substituted aryl group; the process comprising reacting a solution of a compound of formula (2) 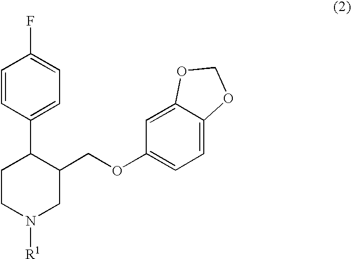
in which R¹ is an alkyl, arylalkyl or alkynyl group; at a temperature in the range of 50-100° C. with a haloformate of formula hal-CO₂R².

2. A process as claimed in claim 1 wherein the solvent is toluene.

3. A process as claimed in claim 1 in which the haloformate is phenyl chloroformate.

4. A process as claimed in claim 3 in which the haloformate is added undiluted.

5. A process as claimed claim 1 wherein phenyl chloroformate is added to a hot toluene solution of the compound of formula (2).

6. A process for the preparation of a carbamate of formula (1) as defined in claim 1 in which R² is phenyl, comprising adding phenyl chloroformate to a solution in toluene of a compound of formula (2) as defined in claim 1; and heating to a temperature in the range of 50-100° C.

7. A process as claimed in claim 1 wherein the temperature is between about 55-75° C.

8. A process as claimed in claim 1 wherein the temperature is 63° C.±5° C.

9. A process as claimed in claim 1 in which the crude carbamate is treated with sulfuric acid as a quenching agent.

10. A process for the preparation of a compound of formula (1) 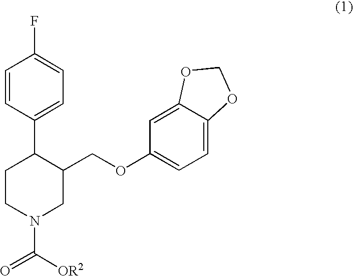
in which R² is a C₁₋₆ alkyl group, optionally substituted by halogen, a C₃₋₆ cycloalkyl group, aralkyl or optionally substituted aryl group; which includes crystallising the compound from propan-2-ol.

11. A process as claimed in claim 10 which involves partition distillation with repeated replacement of distillate with fresh propan-2-ol.

12. A crystalline compound of formula (1) 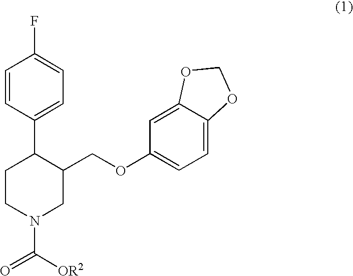
in which R² is a C₁₋₆ alkyl group, optionally substituted by halogen, a C₃₋₆ cycloalkyl group, aralkyl or optionally substituted aryl group.

13. Crystalline 4-(4′-fluorophenyl)-1-phenoxycarbonyl-3-(3′,4′-methylenedioxy phenoxymethyl)-piperidine.

14. A process for preparation of (−)trans4-(4′-fluorophenyl)-3-(3′,4′-methylenedioxy-phenoxymethyl)-piperidine that incorporates the process of claim 1.

15. (−)Trans-4-(4′-fluorophenyl)-3-(3′,4′-methylenedioxy-phenoxymethyl)-piperidine whenever obtained by the process of claim 1.

16. (−)Trans-4-(4′-fluorophenyl)-3-(3′,4′-methylenedioxy-phenoxymethyl)-piperidine as claimed in claim 15, in the form of a hydrochloride or methanesulfonate salt.

17. A method of treating the disorders which comprises administering an effective or prophylactic amount of a compound as claimed in claim 15 to a person suffering from one or more of the Disorders.

18. A method of treating the disorders which comprises administering an effective or prophylactic amount of a compound as claimed in claim 16 to a person suffering from one or more of the Disorders.

19. A process for quenching a crude carbamate of formula (1) 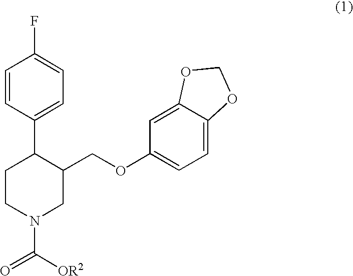
in which R² is a C₁₋₆ alkyl group, optionally substituted by halogen, a C₃₋₆ cycloalkyl group, aralkyl or optionally substituted aryl group; the process comprising treaing the crude carbamate with aqueous sulfuric acid.
